(19) United States
(12) Patent Application Publication
(10) Pub. No.: US 2025/0352367 A1
(43) Pub. Date: Nov. 20, 2025

(54) HELICAL STENT WITH IMPROVED CHARACTERISTICS

(71) Applicant: BOSTON SCIENTIFIC SCIMED, INC., Maple Grove, MN (US)

(72) Inventors: HARRY MULLEADY, Galway (IE); GARY GILMARTIN, Foxford (IE); DANIEL TUCK, Galway (IE); CELINE GLYNN, Galway (IE); JOHN THOMAS O'DRISCOLL, Galway (IE); FIONN STAPLETON, Galway (IE); THOMAS MARTIN KEATING, Galway (IE)

(73) Assignee: BOSTON SCIENTIFIC SCIMED, INC., Maple Grove, MN (US)

(21) Appl. No.: 19/208,943

(22) Filed: May 15, 2025

Related U.S. Application Data
(60) Provisional application No. 63/648,474, filed on May 16, 2024.

(51) Int. Cl. A61F 2/88

(52) U.S. Cl. CPC A61F 2/88; A61F 2230/0069; A61F 2230/0091

(57) ABSTRACT

A medical stent including a tubular scaffold is disclosed. The tubular scaffold is formed from a single wire extending helically from a first end to a second end along a central longitudinal axis such that the single wire forms a plurality of helical windings around the central longitudinal axis. A polymeric covering is disposed on the tubular scaffold and spans gaps between adjacent helical windings of the tubular scaffold. The stent includes a plurality of longitudinal reinforcing strips extending along the tubular scaffold parallel to the central longitudinal axis. The reinforcing strips are configured to restrict elongation of the stent by less than 5% when the stent shifts between a radially collapsed delivery configuration and a radially expanded deployed configuration.

### CROSS-REFERENCE TO RELATED APPLICATIONS

The application claims the benefit of U.S. Provisional Patent Application Ser. No. 63/648,474, filed on May 16, 2024, the disclosure of which is incorporated herein by reference.

### TECHNICAL FIELD

The present disclosure pertains to medical devices, methods for manufacturing medical devices, and uses thereof. More particularly, the present disclosure relates to stents, stent configurations, and methods of manufacture and use of a stent.

### BACKGROUND

An endoprosthesis may be used in the treatment of body lumens. One type of endoprosthesis used in the repair and/or treatment of diseases in various body lumens is a stent. A stent is a generally longitudinal tubular device formed of biocompatible material which is useful to open and support various lumens in the body. For example, stents may be used in the vascular system, urogenital tract, gastrointestinal tract, esophageal tract, tracheal/bronchial tubes, biliary tract, colon, intestine, stomach or other body cavity, as well as in a variety of other applications in the body.

In some instances, it may be desirable to design a stent to include sufficient flexibility and conformability to the body lumen, while maintaining sufficient radial force to open the body lumen at the treatment site and/or prevent migration of the stent within the body lumen. In some instances, it may be desirable to reduce or limit foreshortening. In some instances, different stent configurations may provide different deliverability, flexibility, conformability (e.g., to a body lumen), radial force/strength, and/or anchoring/migration characteristics.

Of the known medical devices and methods, each has certain advantages and disadvantages. There is an ongoing need to provide alternative medical stents as well as alternative methods for manufacturing and using medical stents.

### SUMMARY

This disclosure provides design, material, manufacturing method, and use alternatives for medical devices.

A first example is a medical stent. The stent includes a tubular scaffold extending from a first end to a second end along a central longitudinal axis. The tubular scaffold is formed from a single wire extending helically from the first end to the second end. The single wire forms a plurality of helical windings around the central longitudinal axis. A polymeric covering is disposed on the tubular scaffold and spanning gaps between adjacent helical windings of the tubular scaffold.

Alternatively or additionally to any of the examples herein, in another example, the stent includes a reinforcing filament extending substantially longitudinally along the tubular scaffold.

Alternatively or additionally to any of the examples herein, in another example, the reinforcing filament has a length that is substantially equal to a length of the tubular scaffold.

Alternatively or additionally to any of the examples herein, in another example, the reinforcing filament is interwoven with the plurality of helical windings of the single wire.

Alternatively or additionally to any of the examples herein, in another example, the reinforcing filament is interwoven in an alternating over and under fashion with the plurality of helical windings of the single wire.

Alternatively or additionally to any of the examples herein, in another example, the reinforcing filament is in direct contact with the plurality of helical windings of the single wire.

Alternatively or additionally to any of the examples herein, in another example, the reinforcing filament is an elongated planar strip.

Alternatively or additionally to any of the examples herein, in another example, the reinforcing filament is formed of polyester, polytetrafluoroethylene, or a combination thereof.

Alternatively or additionally to any of the examples herein, in another example, the reinforcing filament is embedded in the polymeric covering.

Alternatively or additionally to any of the examples herein, in another example, a length of the tubular scaffold from the first end to the second end is configured to change by less than 5% when shifting between a radially collapsed delivery configuration and a radially expanded deployed configuration.

Alternatively or additionally to any of the examples herein, in another example, the single wire forms a double helix having a first helical segment of the single wire extending parallel to a second helical segment of the single wire.

Another example is a medical stent. The stent includes a tubular scaffold extending from a first end to a second end along a central longitudinal axis. The tubular scaffold is formed from a single wire extending helically from the first end to the second end. The single wire forms a plurality of helical windings around the central longitudinal axis. A polymeric covering is disposed on the tubular scaffold and spanning gaps between adjacent helical windings of the tubular scaffold. A plurality longitudinal reinforcing strips extend along the tubular scaffold parallel to the central longitudinal axis.

Alternatively or additionally to any of the examples herein, in another example, the plurality of reinforcing strips are interwoven with the plurality of helical windings of the single wire.

Alternatively or additionally to any of the examples herein, in another example, the plurality of reinforcing strips are embedded in the polymeric covering.

Alternatively or additionally to any of the examples herein, in another example, a length of the tubular scaffold from the first end to the second end is configured to change by less than 5% when shifting between a radially collapsed delivery configuration and a radially expanded deployed configuration.

Alternatively or additionally to any of the examples herein, in another example, the single wire forms a double helix having a first helical segment of the single wire extending parallel to a second helical segment of the single wire.

Another example is medical stent. The stent includes a tubular scaffold extending from a first end to a second end along a central longitudinal axis. The tubular scaffold is formed from a single wire extending helically from the first end to the second end. The single wire forms a plurality of helical windings around the central longitudinal axis. A polymeric covering is disposed on the tubular scaffold and spanning gaps between adjacent helical windings of the tubular scaffold. A plurality longitudinal reinforcing strips extend along the tubular scaffold. The plurality of longitudinal reinforcing strips are configured to restrict elongation of the stent by less than 5% when the stent shifts between a radially collapsed delivery configuration and a radially expanded deployed configuration.

Alternatively or additionally to any of the examples herein, in another example, the plurality of reinforcing strips are interwoven with the plurality of helical windings of the single wire.

Alternatively or additionally to any of the examples herein, in another example, the plurality of reinforcing strips are embedded in the polymeric covering.

Alternatively or additionally to any of the examples herein, in another example, the plurality of reinforcing strips are formed of polyester, polytetrafluoroethylene, or a combination thereof.

The above summary of some embodiments, aspects, and/or examples is not intended to describe each disclosed embodiment or every implementation of the present disclosure. The figures and detailed description which follow more particularly exemplify these embodiments.

### BRIEF DESCRIPTION OF THE DRAWINGS

The disclosure may be more completely understood in consideration of the following detailed description in connection with the accompanying drawings, in which:

FIG. 1 is side view illustrating a tubular scaffold of a stent;

FIG. 2 is a side view a stent including the tubular scaffold of FIG. 1;

FIG. 3 is a longitudinal cross-sectional view of the stent of FIG. 2;

FIG. 4 is a side view illustrating an alternative tubular scaffold of a stent; and

FIG. 5 is a longitudinal cross-sectional view of a stent including the tubular scaffold of FIG. 4.

While aspects of the disclosure are amenable to various modifications and alternative forms, specifics thereof have been shown by way of example in the drawings and will be described in detail. It should be understood, however, that the intention is not to limit aspects of the disclosure to the particular embodiments described. On the contrary, the intention is to cover all modifications, equivalents, and alternatives falling within the spirit and scope of the disclosure.

### DETAILED DESCRIPTION

The following description should be read with reference to the drawings, which are not necessarily to scale, wherein like reference numerals indicate like elements throughout the several views. The detailed description and drawings are intended to illustrate but not limit the disclosure. Those skilled in the art will recognize that the various elements described and/or shown may be arranged in various combinations and configurations without departing from the scope of the disclosure. The detailed description and drawings illustrate example embodiments of the disclosure.

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5).

Although some suitable dimensions, ranges, and/or values pertaining to various components, features and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that to facilitate understanding, certain features of the disclosure may be described in the singular, even though those features may be plural or recurring within the disclosed embodiment(s). Each instance of the features may include and/or be encompassed by the singular disclosure(s), unless expressly stated to the contrary. For example, a reference to one feature may be equally referred to all instances and quantities beyond one of said feature unless clearly stated to the contrary. As such, it will be understood that the following discussion may apply equally to any and/or all components for which there are more than one within the device, etc. unless explicitly stated to the contrary.

Relative terms such as “proximal”, “distal”, “advance”, “retract”, variants thereof, and the like, may be generally considered with respect to the positioning, direction, and/or operation of various elements relative to a user/operator/manipulator of the device, wherein “proximal” and “retract” indicate or refer to closer to or toward the user and “distal” and “advance” indicate or refer to farther from or away from the user. In some instances, the terms “proximal” and “distal” may be arbitrarily assigned to facilitate understanding of the disclosure, and such instances will be readily apparent to the skilled artisan. Other relative terms, such as “upstream”, “downstream”, “inflow”, and “outflow” refer to a direction of fluid flow within a lumen, such as a body lumen, a blood vessel, or within a device. Still other relative terms, such as “axial”, “circumferential”, “longitudinal”, “lateral”, “radial”, etc. and/or variants thereof generally refer to direction and/or orientation relative to a central longitudinal axis of the disclosed structure or device.

The term “extent” may be understood to mean the greatest measurement of a stated or identified dimension, unless the extent or dimension in question is preceded by or identified as a “minimum”, which may be understood to mean the smallest measurement of the stated or identified dimension. For example, “outer extent” may be understood to mean an outer dimension, “radial extent” may be understood to mean a radial dimension, “longitudinal extent” may be understood to mean a longitudinal dimension, etc. Each instance of an “extent” may be different (e.g., axial, longitudinal, lateral, radial, circumferential, etc.) and will be apparent to the skilled person from the context of the individual usage. Generally, an “extent” may be considered a greatest possible dimension measured according to the intended usage, while a “minimum extent” may be considered a smallest possible dimension measured according to the intended usage. In some instances, an “extent” may generally be measured orthogonally within a plane and/or cross-section, but may be, as will be apparent from the particular context, measured differently—such as, but not limited to, angularly, radially, circumferentially (e.g., along an arc), etc.

The terms “monolithic” and “unitary” shall generally refer to an element or elements made from or consisting of a single structure or base unit/element. A monolithic and/or unitary element shall exclude structure and/or features made by assembling or otherwise joining multiple discrete structures or elements together.

It is noted that references in the specification to “an embodiment”, “some embodiments”, “other embodiments”, etc., indicate that the embodiment(s) described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it would be within the knowledge of one skilled in the art to implement the particular feature, structure, or characteristic in connection with other embodiments, whether or not explicitly described, unless clearly stated to the contrary. That is, the various individual elements described below, even if not explicitly shown in a particular combination, are nevertheless contemplated as being combinable or arrangeable with each other to form other additional embodiments or to complement and/or enrich the described embodiment(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. It is to be understood that the numerical nomenclature is not intended to be limiting and is exemplary only. In some embodiments, alterations of and deviations from previously used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

Additionally, it should be noted that in any given figure, some features may not be shown, or may be shown schematically, for clarity and/or simplicity. Additional details regarding some components and/or method steps may be illustrated in other figures in greater detail. It is noted that some reference numbers may be discussed but are not expressly shown with respect to a particular figure. Reference numbers discussed but not expressly shown may be shown in other figures. Similarly, some reference numbers shown but not expressly discussed may be discussed with respect to other figures herein. The systems, devices, and/or methods disclosed herein may provide a number of desirable features and benefits as described in more detail below.

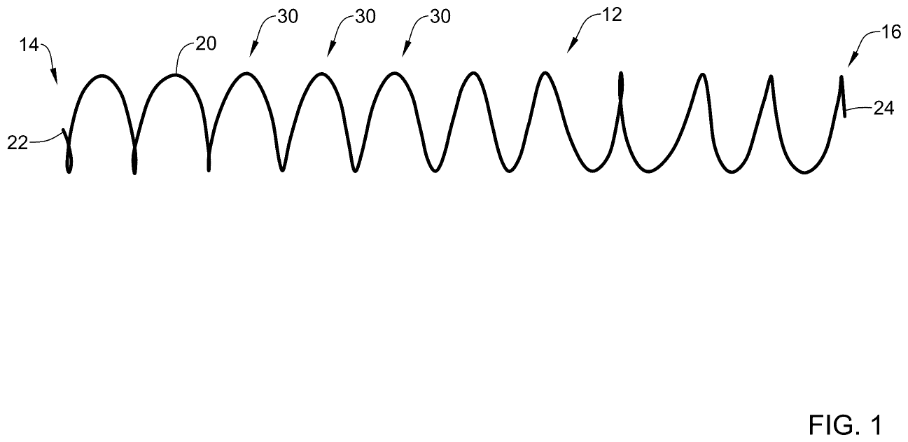

FIG. 1 depicts a side view of a tubular scaffold 12 of a stent 10 according to examples of the present disclosure. In this and other examples, the tubular scaffold 12 has a first end 14, a second end 16, and a body extending therebetween. The tubular scaffold 12 may define a lumen extending through the tubular scaffold 12, and thus the stent 10, from the first end 14 to the second end 16. The tubular scaffold 12 may be formed from a single wire 20, and the single wire 20 may be shaped into a generally helical coil to form a plurality of coil windings 30 throughout the body of the tubular scaffold 12. Each of the plurality of windings 30 may be a single complete revolution (i.e., 360 degrees) of the wire 20 about a central longitudinal axis of the tubular scaffold 12 as the wire 20 extends in a helical direction along the length of the tubular scaffold 12. The single wire 20 may have a first end (i.e., first terminal end) 22 of the wire 20 proximate the first end 14 of the tubular scaffold 12 and a second end (i.e., second terminal end) 24 of the wire 20 proximate the second end 16 of the tubular scaffold 12.

Accordingly, the tubular scaffold 12 may be formed of a plurality of helical windings 30 of the wire 20 in which the wire 20 extends helically along the entire length of the tubular scaffold 12 from the first terminal end 22 of the wire 20 to the second terminal end 24 of the wire 20. The tubular scaffold 12 may include any desired number of helical windings 30, depending on the spacing between adjacent windings 30 and/or the overall length of the tubular scaffold 12, and thus the stent 10.

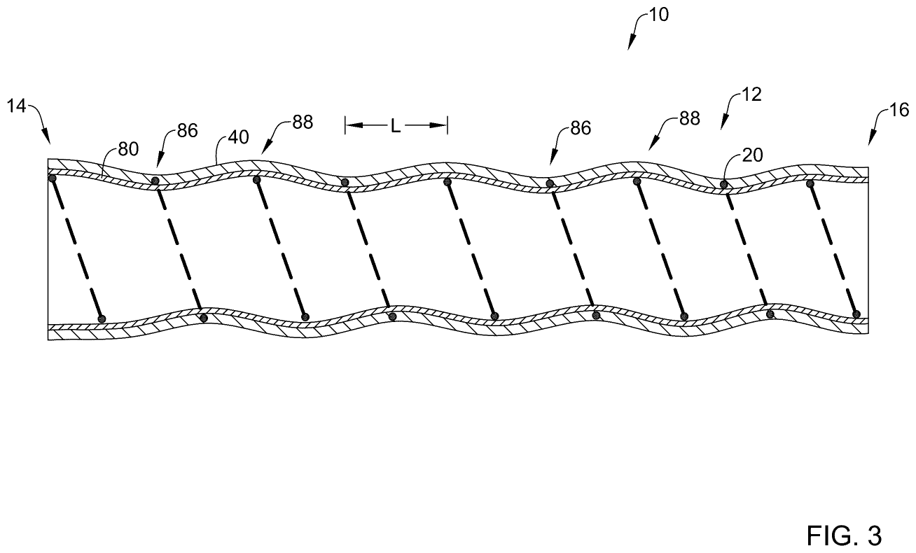

The axial spacing L (see FIG. 3) between adjacent helical windings 30 of the plurality of helical windings may be uniform along the length of the tubular scaffold 12. For example, in some instances the axial spacing L may be in the range of about 2 millimeters to about 15 millimeters, in the range of about 2 millimeters to about 10 millimeters, in the range of about 5 millimeters to about 10 millimeters, or in the range of about 5 millimeters to about 15 millimeters. In some embodiments, the axial spacing L between adjacent helical windings 30 of the plurality of helical windings may vary along the length of the tubular scaffold 12. In some embodiments, the axial spacing L between adjacent helical windings 30 of the plurality of helical windings may increase along the length of the tubular scaffold 12 from the first end 14 toward and/or to the second end 16. Alternatively, in some embodiments, the axial spacing L between adjacent helical windings 30 of the plurality of helical windings may increase along the length of the tubular scaffold 12 from the second end 12 toward and/or to the first end 14. In other embodiments, the axial spacing L between adjacent helical windings 30 of the plurality of helical windings may increase along the length of the tubular scaffold 12 from both the first end 14 and the second end 16 toward the middle of the tubular scaffold 12. In yet other embodiments, the axial spacing L between adjacent helical windings 30 of the plurality of helical windings may decrease along the length of the tubular scaffold 12 from both the first end 14 and the second end 16 toward the middle of the tubular scaffold 12. Other configurations are also contemplated.

The wire 20 of the tubular scaffold 12 of the stent 10 may be formed from one or more suitable materials. Example suitable materials include, but are not limited to, metals, metal alloys, shape memory alloys, polymers, nickel-titanium alloys, cobalt-chromium-nickel-molybdenum alloys, and/or other suitable materials enabling the tubular scaffold 12, and thus the stent 10, to be radially expanded into a shape when positioned at a target site. In some instances, the material may be selected to enable the stent 10 to be removed with relative ease as well. In some examples, the wire 20 may be formed from alloys such as, but not limited to, nitinol and/or Elgiloy®.

In some embodiments, the outer diameter of the body portion may be generally uniform and/or generally constant along the length of the tubular scaffold 12 and/or the stent 10. In some embodiments, the outer diameter of the body portion may be generally uniform and/or generally constant along the length of the body portion except for a flared first end portion and/or a flared second end portion, where present, wherein an outer diameter of the flared first end portion is greater than an outer diameter of the body portion and/or an outer diameter of the flared second end portion is greater than the outer diameter of the body portion. In some embodiments, the outer diameter of the body portion may be generally uniform and/or generally constant along the length of the body portion from a flared first end portion to a flared second end portion, where present. Other configurations are also contemplated. The flared first end portion may be disposed proximate and/or adjacent to the first end 14. In some embodiments, the flared first end portion may extend from the first end 14 to the body portion. The flared second end portion may be disposed proximate and/or adjacent to the second end 16. In some embodiments, the flared second end portion may extend from the second end 16 to the body portion.

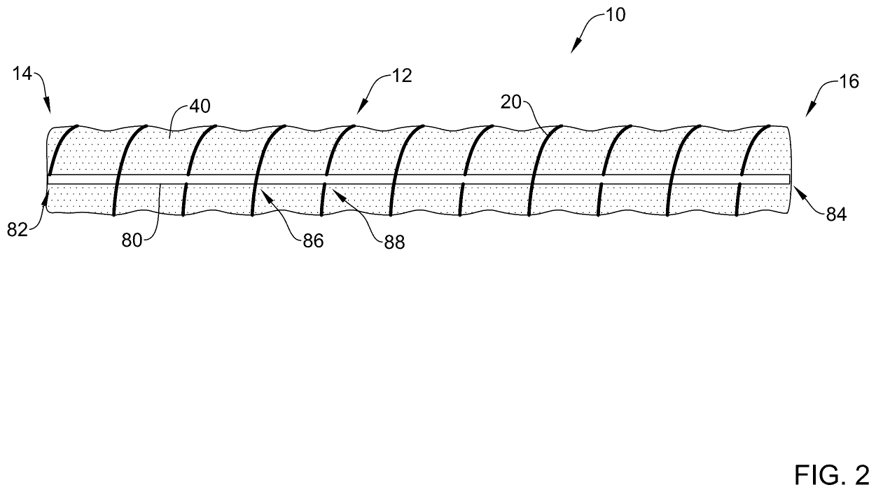

In various embodiments, the tubular scaffold 12 of the stent 10 may be partially or fully covered, uncovered, coated, or a combination thereof. Various stent embodiments described herein may include a full or partial covering, coating, or other membrane over an interior surface of the tubular scaffold 12 and/or over an exterior surface of the tubular scaffold 12. FIG. 2 illustrates the stent 10 including a covering 40, such as a polymeric covering, applied to the tubular scaffold 12 and extending along an entire length of the stent 10. The covering 40 may extend across the gap between adjacent helical windings 30 of the tubular scaffold 12. The covering 40 (e.g., coating, or other membrane) may comprise polymeric material, such as silicone, polyurethane, or other desired material. Some additional materials include, but are not limited to, polytetrafluoroethylene, expanded polytetrafluoroethylene, polyvinylidene fluoride, an aromatic polycarbonate-based thermoplastic urethane, and/or other like materials. In some instances, the covering 40 may include ingrowth promoting materials for interfacing with tissue.

The wire 20 may be partially or fully embedded within the covering 40, and in some instances the wire 20 may be fully encapsulated by the covering 40. The covering 40 may be applied by dip coating, roll coating, painting, spraying, other known disposition method, or a combination thereof. The covering 40 may inhibit tissue growth into the lumen of the stent 10 and/or minimize fluid leakage from within and/or without the stent 10. By limiting tissue ingrowth, the stent 10 is less likely to become occluded with organic matter and allows proper perfusion and flow of biological fluids through the stent 10 during its life of implantation. Limiting tissue ingrowth may also result in less traumatic removal of the stent 10.

The covering 40 (e.g., coating), when applied to the tubular scaffold 12 of the stent 10, may be applied to any suitable portion of the stent 10. The covering 40 (e.g., coating) may be applied to an entirety of the body of the tubular scaffold 12 of the stent 10, but this is not required and the covering 40 (e.g., coating) may be applied to only a portion of the tubular scaffold 12 of the stent 10 that is less than the entirety of the stent 10, if desired. For example, the covering 40 may not extend an entire length of the stent 10, leaving portions of the tubular scaffold 12 uncovered and devoid of the covering 40.

The stent 10 may comprise one or more, or a plurality of reinforcing filaments 80. In some embodiments, the reinforcing filament(s) 80 may be formed of polyester, polytetrafluoroethylene (PTFE), or a combination thereof, among other suitable materials such as those detailed herein. In some embodiments, the reinforcing filament(s) 80 may be formed of polyester. In some embodiments, the reinforcing filament(s) 80 may be formed of PTFE.

The reinforcing filament(s) 80 may have a uniform width taken along an entire length (e.g., extending longitudinally along the central longitudinal axis of the stent 10), as illustrated in FIG. 2. However, in some embodiments, the reinforcing filament(s) 80 can have a variable width (e.g., different widths taken at two or more positions along the length of the reinforcing filament 80).

The reinforcing filament 80 may have a uniform width and/or uniform thickness. For instance, the reinforcing filament 80 may have a uniform thickness (e.g., in the direction normal to the central longitudinal axis of the stent 10) along an entire length (e.g., in a direction along or coaxial with the central longitudinal axis of the stent) of the reinforcing filament 80. However, in some embodiments, the reinforcing filament 80 can have a variable thickness (e.g., different thicknesses at two or more positions along the length of the reinforcing filament 80).

The reinforcing filament 80 may be formed of a different material than a material of another portion of the stent 10 (e.g., a different than a material of the tubular scaffold 12 and/or than a material of the polymeric covering 40). For instance, the reinforcing filament 80 may be formed of a different material than the covering 40. Thus, the reinforcing filament 80 may have a different material property (e.g., a higher tensile strength) than a corresponding material property of the covering 40 of the stent 10. Thus, the presence of the reinforcing filament 80 may alter the overall properties of the stent 10, for example, increasing an overall tensile strength of the stent 10, thereby improving the characteristics of the stent 10 (e.g., mitigating any foreshortening of the stent 10 during deployment of the stent 10). For example, the presence of the reinforcing filament 80 may prevent any elongation when the stent 10 is constrained for delivery, and thereby prevent any subsequent foreshortening of the stent 10 when the stent 10 is deployed and radially expanded at a treatment site in a body lumen.

In some embodiments, the reinforcing filament 80 may have a tensile strength of about 500 pounds per square inch or more, about 1,000 pounds per square inch or more, about 1,500 pounds per square inch or more, or about 2,000 pounds per square inch or more, for example. In certain embodiments, the reinforcing filament 80 may possess a tensile strength several orders of magnitude greater than that of the polymeric covering 40. For instance, the reinforcing filament 80 could exhibit a tensile strength at least two, three, four, five, six, seven, eight, nine, or ten times greater than that of the polymeric covering 40. For example, in some embodiments, the tensile strength of the reinforcing filament 80 may be in a range from about 2,000 to about 3,000 pounds per square inch, while that of the polymeric covering 40 may be in a range from about 200 to about 300 pounds per square inch, among other possibilities.

In some embodiments, the reinforcing filament 80 may be manifested as an elongated planar strip, as illustrated in FIG. 2. Having the reinforcing filament 80 be manifested as an elongate planar strip can promote aspects herein (e.g., mitigation of any foreshortening experienced by the stent 10), and yet can maintain a relatively small or thin profile of the stent 10. However, other configurations of the reinforcing filament 80 are possible. For instance, in some embodiments, the reinforcing filament 80 can be manifested as an elongate rod or other structure that can be interwoven with the helical windings 30 of the single wire 20 of the tubular scaffold 12.

The presence of the reinforcing filament 80 can promote aspects herein such as promoting mitigation of any foreshortening experienced by the stent 10, promoting conformability (e.g., to body lumen) of the stent 10, and/or mitigating any forces (e.g., axial and/or radial forces) imparted on the stent 10. As illustrated in FIG. 2, the reinforcing filament 80 can extend substantially longitudinally along the stent 10 from the first end 14 to the second end 16. For instance, the reinforcing filament 80 may extend continuously and substantially longitudinally along the tubular scaffold 12, and in some instances coterminous with the tubular scaffold 12. For example, in some embodiments the reinforcing filament 80 may have a length that is substantially equal to at least half a length of the tubular scaffold 12. However, in some embodiments the reinforcing filament 80 may have a length that is substantially equal to a length of the tubular scaffold 12, as illustrated in FIG. 2. Having a length of the reinforcing filament 80 be substantially equal to a length of the tubular scaffold 12 can promote aspects herein such as promoting mitigation of any foreshortening experienced by the stent 10, promoting conformability of the stent 10, and/or mitigating any forces (e.g., axial and/or radial forces) imparted on the stent 10 along an entire length of the stent 10.

In some embodiments the reinforcing filament(s) 80 may extend along an exterior of the tubular scaffold 12 (i.e., radially outward of the tubular scaffold 12) along an entire length of the tubular scaffold 12. In other embodiments, the reinforcing filament(s) 80 may extend along an interior of the tubular scaffold 12 (i.e., radially inward of the tubular scaffold 12) along an entire length of the tubular scaffold 12. In some embodiments, the reinforcing filament 80 may be interwoven with the single wire 20 forming the tubular scaffold 12. For example, the reinforcing filament 80 may be interwoven with two or more of the plurality of helical windings 30 of the single wire 20. For instance, as illustrated in FIG. 2, and as described in greater detail in FIG. 3, the reinforcing filament 80 may be interwoven in an alternating over and under fashion with the plurality of helical windings 30 of the single wire 20. For example, as detailed in FIG. 3 the reinforcing filament 80 may be pass along (e.g., be in direct contact with) a given side (e.g., a radially interior side) of a first helical winding 30 at a first intersection 86 of the reinforcing filament 80 and the single wire 20, and may pass substantially longitudinally along (e.g., be in direct contact with) a different side (e.g., a radially exterior side) of a second helical winding 30 at a second intersection 88 of the reinforcing filament 80 and the single wire 20 that is longitudinally adjacent to the first intersection 86. The reinforcing filament 80 can continue to pass substantially longitudinally along a side of additional helical windings 30 in this alternating over and under fashion. For instance, the reinforcing filament 80 can pass along each of the helical windings 30 where the reinforcing filament 80 intersects the single wire 20 in an alternating over and under fashion. Having the reinforcing filament 80 pass along each of the helical windings 30 in an alternating over and under fashion can promote aspects herein such as promoting mitigation of any foreshortening experienced by the stent 10, promoting conformability of the stent 10, and/or mitigating any forces (e.g., axial and/or radial forces) imparted on the stent 10 along an entire length of the stent 10. However, other configurations are possible. For instance, the reinforcing filament 80 may pass by the same respective side (e.g., a radially interior) of two or more consecutive of helical windings 30 of the single wire 20 prior to passing by a different side (e.g., a radially exterior) of another of one or more (e.g., two or more consecutive helical windings 30) of the helical windings 30 of the single wire 20. That is the reinforcing filament 80 can pass “over” a first subset of the helical windings 30 of the single wire 20 and can pass “under” a second subset (e.g., some or all) of the remaining helical windings 30 of the single wire 20. In any case, interweaving the reinforcing filament 80 with some or all of the plurality of the helical windings 30 the single wire 20 can promote aspects herein.

FIG. 3 is a longitudinal cross-sectional view of the stent 10 of FIG. 2. In some embodiments, the reinforcing filament 80 can be in direct contact with the single wire 20 such as being in direct contact with the plurality of helical windings 30 as the reinforcing filament 80 crosses the single wire 20. For example, the reinforcing filament 80 can be in direct contact with the single wire 20 at the intersections 86, 88 of the reinforcing filament 80 and the single wire 20. As used herein, “direct” contact refers to the absence of the intervening materials. For instance, as illustrated in FIG. 3, the reinforcing filament 80 can be in direct contact with an outer surface of the single wire 20 at the intersections 86, 88.

While FIG. 2 illustrates the presence of an individual reinforcing filament 80, in some embodiments, the stent 10 can include a plurality of reinforcing filaments 80. For instance, FIG. 3, illustrates the stent 10 including a plurality of reinforcing filaments 80, on opposing sides of the tubular scaffold 12. Additional reinforcing filaments 80 are contemplated, if desired. As illustrated in FIG. 3, each of the plurality of reinforcing filaments 80 can extend substantially longitudinally along the tubular scaffold 12. For instance, each of the plurality of reinforcing filaments 80 may extend parallel to a central longitudinal axis of the stent 10. However, in some embodiments a reinforcing filament may extend at an angle (e.g., helically) or be transverse to the central longitudinal axis. For instance, in some embodiments some or all of the reinforcing filaments may pass helically along the tubular scaffold 12, among other possibilities.

When a plurality of reinforcing filaments 80 are included in the stent 10, the plurality of reinforcing filaments 80 may be spaced apart circumferentially about the tubular scaffold 12. For instance, each of the reinforcing filaments 80 may be spaced apart circumferentially from an adjacent reinforcing filament 80, among other possible configurations. For instance, the plurality of reinforcing filaments 80 may include a first reinforcing filament 80 and a second reinforcing filament 80, where the first reinforcing filament 80 and the second reinforcing filament 80 may be equally spaced apart circumferentially about the tubular scaffold 12 by about 180 degrees.

In some embodiments, the first reinforcing filament 80 and the second reinforcing filament 80 can be interwoven with corresponding portions of the plurality of helical windings 30 of the tubular scaffold 12 in the same manner. However, in some embodiments, the first reinforcing filament 80 and the second reinforcing filament 80 can be interwoven with corresponding portions of the plurality of helical windings 30 of the tubular scaffold 12 in a different manner.

In some embodiments, the plurality of reinforcing filaments 80 may be the same shape, the same size, or both the same shape and the same size. Having the plurality of reinforcing filaments 80 be the same size, same shape, or both, can promote aspects herein. For instance, in some embodiments, each of the plurality of reinforcing filaments 80 may be the same shape and same size (e.g., may have the same length along the central longitudinal axis of the stent 10, same width along a direction that is traverse to the central longitudinal axis of the stent 10, and may have the same thickness). However, in some embodiments one or more of the plurality of reinforcing filaments 80 may have a different shape and/or size than another one of the plurality of reinforcing filaments 80. In some embodiments, each of the plurality of reinforcing filaments 80 may be interwoven with the plurality of helical windings 30, as shown in FIG. 3.

In some embodiments, the stent 10 may be configured to shift between a radially constrained delivery configuration, and a radially expanded deployed configuration, when unconstrained. In some embodiments, the stent 10 may be configured to self-expand from the radially constrained delivery configuration to the radially expanded deployed configuration, when a constrainment force is removed from the stent 10. That is, the stent 10 may be a self-expanding device such as known or heretofore known to those of ordinary skill in the art. For instance, the tubular scaffold 12 may be formed of shape-memory or heat-formable material (e.g., Nitinol or Elgiloy® or shape memory polymers) so that the tubular scaffold 12 returns to a pre-shaped radially expanded configuration from a radially constrained configuration upon deployment from a delivery sheath (any acceptable tubular elongated member such as known to those of ordinary skill in the art for delivery of medical devices) and/or withdrawal of a delivery sheath which maintains the stent 10 in a delivery configuration therein.

In some embodiments, the reinforcing filament(s) 80 may be configured to minimize and/or avoid foreshortening and/or a change in the length of the tubular scaffold 12 and/or the stent 10 as the tubular scaffold 12 and/or the stent 10 shifts between the radially collapsed delivery configuration and the radially expanded deployed configuration. Thus, the reinforcing filament(s) 80 may permit the expandable scaffold 12 and/or stent 10 to change diameter (between the radially collapsed configuration and the radially expanded configuration) while restricting axial elongation and/or axial contraction of the expandable scaffold 12 and/or stent 10. In some embodiments, the length of the expandable scaffold 12 and/or the stent 10 from the first end 14 to the second end 16 may be configured to change by less than 10% when shifting between the radially collapsed delivery configuration and the radially expanded deployed configuration. In some embodiments, the length of the tubular scaffold 12 and/or the stent 10 from the first end 14 to the second end 16 may be configured to change by less than 7.5% when shifting between the radially collapsed delivery configuration and the radially expanded deployed configuration. In some embodiments, the length of the tubular scaffold 12 and/or the stent 10 from the first end 14 to the second end 16 may be configured to change by less than 5% when shifting between the radially collapsed delivery configuration and the radially expanded deployed configuration. In some embodiments, the length of the tubular scaffold 12 and/or the stent 10 from the first end 14 to the second end 16 may be configured to change by less than 2.5% when shifting between the radially collapsed delivery configuration and the radially expanded deployed configuration. In some embodiments, the length of the tubular scaffold 12 and/or the stent 10 from the first end 14 to the second end 16 may be configured to change by less than 1% when shifting between the radially collapsed delivery configuration and the radially expanded deployed configuration. Other configurations are also contemplated.

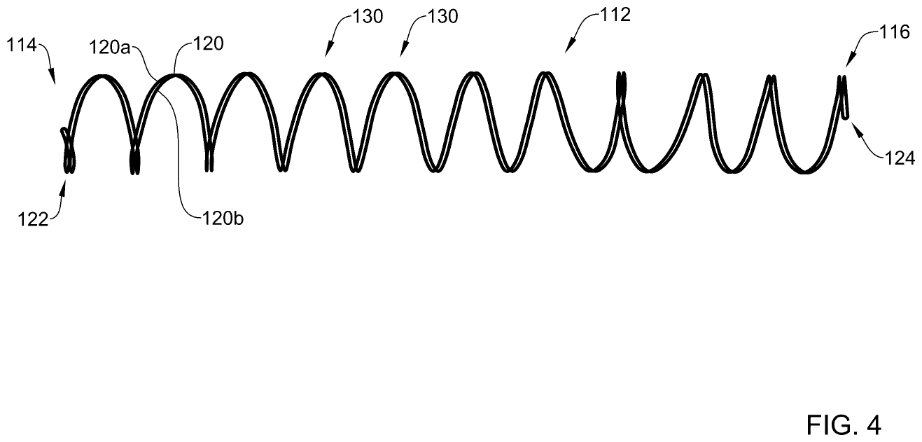

FIG. 4 depicts a side view of an alternative configuration of a tubular scaffold 112 of a stent 110 according to examples of the present disclosure. In this and other examples, the tubular scaffold 112 has a first end 114, a second end 116, and a body extending therebetween. The tubular scaffold 112 may define a lumen extending through the tubular scaffold 112, and thus the stent 110, from the first end 114 to the second end 116. The tubular scaffold 112 may be formed from a single wire 120, and the single wire 120 may be shaped into a generally double helical coil to form a plurality of coil windings 130 throughout the body of the tubular scaffold 112. Each of the plurality of windings 130 may be a single complete revolution (i.e., 360 degrees) of two parallel segments of the wire 120 about a central longitudinal axis of the tubular scaffold 112 as the two parallel segments of the wire 120 extend in a helical direction along the length of the tubular scaffold 112. The single wire 120 may be a continuous length of wire (i.e., with wire ends welded or otherwise joined together to form a continuous loop of wire) having a bend at a first end 122 proximate the first end 114 of the tubular scaffold 112 and a bend at a second end 124 of the proximate the second end 116 of the tubular scaffold 112, such that two parallel segments of the wire 120 (e.g., a first segment 120a and a second segment 120b) extend in a helical direction parallel to one another between the first bend at the first end 122 and the second bend at the second end 124. Accordingly, the tubular scaffold 112 may be formed of a plurality of helical windings 130 of the wire 120 in which two parallel segments of the wire 120 (e.g., a first segment 120a and a second segment 120b) extend helically along the entire length of the tubular scaffold 112. The tubular scaffold 112 may include any desired number of helical windings 130, depending on the spacing between adjacent windings 130 and/or the overall length of the tubular scaffold 112, and thus the stent 110.

The axial spacing between adjacent helical windings 130 of the plurality of helical windings may be uniform along the length of the tubular scaffold 112, or the axial spacing between adjacent helical windings 130 may vary along the length of the tubular scaffold 112, as described above. Furthermore, the two parallel segments of the wire 120 (e.g., a first segment 120a and a second segment 120b) may be at any axial distance apart. In some instances, the two parallel segments of the wire 120 may directly adjacent to one another such that the first segment 120a is in substantially direct contact with the second segment 120b along the helical pathway. In other instances, the two parallel segments of the wire 120 may be spaced apart from one another such that there is a helical gap extending between the first segment 120a and the second segment 120b.

The wire 120 of the tubular scaffold 112 of the stent 110 may be formed from one or more suitable materials. Example suitable materials include, but are not limited to, metals, metal alloys, shape memory alloys, polymers, nickel-titanium alloys, cobalt-chromium-nickel-molybdenum alloys, and/or other suitable materials enabling the tubular scaffold 112, and thus the stent 110, to be radially expanded into a shape when positioned at a target site. In some instances, the material may be selected to enable the stent 110 to be removed with relative ease as well. In some examples, the wire 120 may be formed from alloys such as, but not limited to, nitinol and/or Elgiloy®.

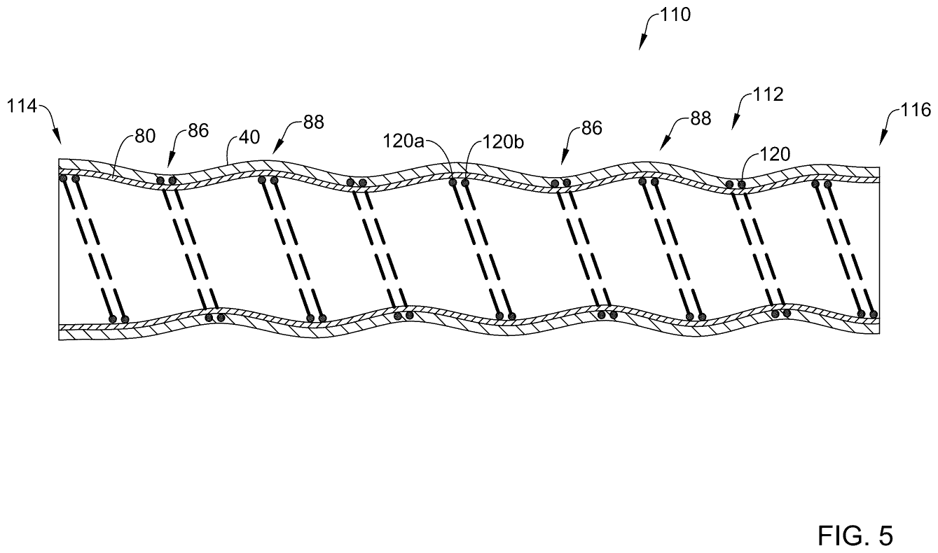

FIG. 5 is a longitudinal cross-sectional view of a stent 110 including the tubular scaffold 112 of FIG. 4 and a covering 40 applied therewith. In various embodiments, the tubular scaffold 112 of the stent 110 may be partially or fully covered, uncovered, coated, or a combination thereof. Various stent embodiments described herein may include a full or partial covering, coating, or other membrane over an interior surface of the tubular scaffold 112 and/or over an exterior surface of the tubular scaffold 112. FIG. 5 illustrates the stent 110 including a covering 40, such as a polymeric covering, applied to the tubular scaffold 112 and extending along an entire length of the stent 110. The covering 40 may extend across the gap between adjacent helical windings 130 of the tubular scaffold 112. The covering 40 (e.g., coating, or other membrane) may comprise polymeric material, such as silicone, polyurethane, or other desired material. Some additional materials include, but are not limited to, polytetrafluoroethylene, expanded polytetrafluoroethylene, polyvinylidene fluoride, an aromatic polycarbonate-based thermoplastic urethane, and/or other like materials. In some instances the covering 40 may include ingrowth promoting materials for interfacing with tissue.

The wire 120 may be partially or fully embedded within the covering 40, and in some instances the wire 120 may be fully encapsulated by the covering 40. The covering 40 may be applied by dip coating, roll coating, painting, spraying, other known disposition method, or a combination thereof. The covering 40 may inhibit tissue growth into the lumen of the stent 110 and/or minimize fluid leakage from within and/or without the stent 110. By limiting tissue ingrowth, the stent 110 is less likely to become occluded with organic matter and allows proper perfusion and flow of biological fluids through the stent 110 during its life of implantation. Limiting tissue ingrowth may also result in less traumatic removal of the stent 110.

The covering 40 (e.g., coating), when applied to the tubular scaffold 112 of the stent 110, may be applied to any suitable portion of the stent 110. The covering 40 (e.g., coating) may be applied to an entirety of the body of the tubular scaffold 112 of the stent 110, but this is not required and the covering 40 (e.g., coating) may be applied to only a portion of the tubular scaffold 112 of the stent 110 that is less than the entirety of the stent 110, if desired. For example, the covering 40 may not extend an entire length of the stent 110, leaving portions of the tubular scaffold 112 uncovered and devoid of the covering 40.

As further shown in FIG. 5, the stent 110 may comprise one or more, or a plurality of reinforcing filaments 80, similar to that described above. In some embodiments, the reinforcing filament(s) 80 may be formed of polyester, polytetrafluoroethylene (PTFE), or a combination thereof, among other suitable materials such as those detailed herein. In some embodiments, the reinforcing filament(s) 80 may be formed of polyester. In some embodiments, the reinforcing filament(s) 80 may be formed of PTFE.

The reinforcing filament(s) 80 may have any suitable shape and/or configuration, such as described herein. In some instances, the reinforcing filament(s) 80 may have a uniform width taken along an entire length (e.g., extending longitudinally along the central longitudinal axis of the stent 110). However, in some embodiments, the reinforcing filament(s) 80 can have a variable width (e.g., different widths taken at two or more positions along the length of the reinforcing filament 80).

The reinforcing filament(s) 80 may be formed of a different material than a material of another portion of the stent 110 (e.g., a different than a material of the tubular scaffold 112 and/or than a material of the polymeric covering 40). For instance, the reinforcing filament(s) 80 may be formed of a different material than the covering 40. Thus, the reinforcing filament(s) 80 may have a different material property (e.g., a higher tensile strength) than a corresponding material property of the covering 40 of the stent 110. Thus, the presence of the reinforcing filament(s) 80 may alter the overall properties of the stent 110, for example, increasing an overall tensile strength of the stent 110, thereby improving the characteristics of the stent 110 (e.g., mitigating any foreshortening of the stent 110 during deployment of the stent 110). For example, the presence of the reinforcing filament(s) 80 may prevent any elongation when the stent 110 is constrained for delivery, and thereby prevent any subsequent foreshortening of the stent 110 when the stent 110 is deployed and radially expanded at a treatment site in a body lumen.

In some embodiments, the reinforcing filament(s) 80 may have a tensile strength of about 500 pounds per square inch or more, about 1,000 pounds per square inch or more, about 1,500 pounds per square inch or more, or about 2,000 pounds per square inch or more, for example. In certain embodiments, the reinforcing filament(s) 80 may possess a tensile strength several orders of magnitude greater than that of the polymeric covering 40. For instance, the reinforcing filament(s) 80 could exhibit a tensile strength at least two, three, four, five, six, seven, eight, nine, or ten times greater than that of the polymeric covering 40. For example, in some embodiments, the tensile strength of the reinforcing filament(s) 80 may be in a range from about 2,000 to about 3,000 pounds per square inch, while that of the polymeric covering 40 may be in a range from about 200 to about 300 pounds per square inch, among other possibilities.

In some embodiments, the reinforcing filament(s) 80 may be manifested as an elongated planar strip, as described elsewhere herein. Having the reinforcing filament(s) 80 be manifested as an elongate planar strip can promote aspects herein (e.g., mitigation of any foreshortening experienced by the stent 110), and yet can maintain a relatively small or thin profile of the stent 110. However, other configurations of the reinforcing filament(s) 80 are possible. For instance, in some embodiments, the reinforcing filament(s) 80 can be manifested as an elongate rod or other structure that can be interwoven with the helical windings 130 of the single wire 120 of the tubular scaffold 112.

The presence of the reinforcing filament(s) 80 can promote aspects described herein, such as promoting mitigation of any foreshortening experienced by the stent 110. The reinforcing filament(s) 80 can extend substantially longitudinally along the stent 110 from the first end 114 to the second end 116. For instance, the reinforcing filament(s) 80 may extend continuously and substantially longitudinally along the tubular scaffold 112, and in some instances coterminous with the tubular scaffold 112. For example, in some embodiments the reinforcing filament(s) 80 may have a length that is substantially equal to at least half a length of the tubular scaffold 112. However, in some embodiments the reinforcing filament(s) 80 may have a length that is substantially equal to a length of the tubular scaffold 112. Having a length of the reinforcing filament 80 be substantially equal to a length of the tubular scaffold 112 can promote aspects herein such as promoting mitigation of any foreshortening experienced by the stent 110, promoting conformability of the stent 110, and/or mitigating any forces (e.g., axial and/or radial forces) imparted on the stent 110 along an entire length of the stent 110.

In some embodiments the reinforcing filament(s) 80 may extend along an exterior of the tubular scaffold 112 (i.e., radially outward of the tubular scaffold 12) along an entire length of the tubular scaffold 112. In other embodiments, the reinforcing filament(s) 80 may extend along an interior of the tubular scaffold 112 (i.e., radially inward of the tubular scaffold 12) along an entire length of the tubular scaffold 112. In some embodiments, the reinforcing filament 80 may be interwoven with the double helical wire 120 forming the tubular scaffold 112. For example, the reinforcing filament(s) 80 may be interwoven with two or more of the plurality of helical windings 130 of the double helical wire 120. For instance, as illustrated in FIG. 5, the reinforcing filament(s) 80 may be interwoven in an alternating over and under fashion with the plurality of helical windings 130 of the double helical wire 120. For example, as detailed in FIG. 5 the reinforcing filament(s) 80 may be pass along (e.g., be in direct contact with) a given side (e.g., a radially interior side) of the first segment 120a and second segment 120b of a first helical winding 130 at a first intersection 86 of the reinforcing filament 80 and the double helical wire 120, and may pass substantially longitudinally along (e.g., be in direct contact with) a different side (e.g., a radially exterior side) of the first segment 120a and second segment 120b of a second helical winding 130 at a second intersection 88 of the reinforcing filament 80 and the double helical wire 120 that is longitudinally adjacent to the first intersection 86. The reinforcing filament 80 can continue to pass substantially longitudinally along a side of additional helical windings 130 in this alternating over and under fashion. For instance, the reinforcing filament 80 can pass along each of the helical windings 130 where the reinforcing filament 80 intersects the double helical wire 120 in an alternating over and under fashion. However, other configurations are possible. For instance, in other instances the reinforcing filament 80 may pass between the first segment 120a and the second segment 120b of the double wire 120 at one or more, or at each intersection of the reinforcing filament 80 with the helical windings 130 of the double helical wire 120. That is the reinforcing filament 80 can pass “over” the first segment 120a of the double helical wire 130 and can pass “under” the second segment 120b of the double helical wire 130 at one or more, or at each intersection of the reinforcing filament 80 with the helical windings 130 of the double helical wire 120. In any case, interweaving the reinforcing filament 80 with some or all of the plurality of the helical windings 130 the wire 120 can promote aspects herein.

In some embodiments, the reinforcing filament(s) 80 can be in direct contact with the single wire 120 such as being in direct contact with the plurality of double helical windings 130 as the reinforcing filament 80 crosses the single wire 120. For example, the reinforcing filament 80 can be in direct contact with the single wire 120 at the intersections 86, 88 of the reinforcing filament 80 and the single wire 120. As used herein, “direct” contact refers to the absence of the intervening materials. For instance, as illustrated in FIG. 5, the reinforcing filament 80 can be in direct contact with an outer surface of the single wire 120 at the intersections 86, 88.

The stent 110 can include any quantity of reinforcing filaments 80 desired. For instance, the stent 110 can include first and second reinforcing filaments 80, on opposing sides of the tubular scaffold 112. Additional reinforcing filaments 80 are contemplated, if desired. As illustrated in FIG. 5, each of the plurality of reinforcing filaments 80 can extend substantially longitudinally along the tubular scaffold 112. For instance, each of the plurality of reinforcing filaments 80 may extend parallel to a central longitudinal axis of the stent 110. However, in some embodiments a reinforcing filament may extend at an angle (e.g., helically) or be transverse to the central longitudinal axis. For instance, in some embodiments some or all of the reinforcing filaments may pass helically along the tubular scaffold 112, among other possibilities.

When a plurality of reinforcing filaments 80 are included in the stent 110, the plurality of reinforcing filaments 80 may be spaced apart circumferentially about the tubular scaffold 112. For instance, each of the reinforcing filaments 80 may be spaced apart circumferentially from an adjacent reinforcing filament 80, among other possible configurations. For instance, the plurality of reinforcing filaments 80 may include a first reinforcing filament 80 and a second reinforcing filament 80, where the first reinforcing filament 80 and the second reinforcing filament 80 may be equally spaced apart circumferentially about the tubular scaffold 112 by about 180 degrees.

In some embodiments, the stent 110 may be configured to shift between a radially constrained delivery configuration, and a radially expanded deployed configuration, when unconstrained. In some embodiments, the stent 110 may be configured to self-expand from the radially constrained delivery configuration to the radially expanded deployed configuration, when a constrainment force is removed from the stent 10.

In some embodiments, the reinforcing filament(s) 80 may be configured to minimize and/or avoid foreshortening and/or a change in the length of the tubular scaffold 112 and/or the stent 110 as the tubular scaffold 112 and/or the stent 110 shifts between the radially collapsed delivery configuration and the radially expanded deployed configuration. Thus, the reinforcing filament(s) 80 may permit the expandable scaffold 112 and/or stent 110 to change diameter (between the radially collapsed configuration and the radially expanded configuration) while restricting axial elongation and/or axial contraction of the expandable scaffold 112 and/or stent 110. In some embodiments, the length of the expandable scaffold 112 and/or the stent 110 from the first end 114 to the second end 116 may be configured to change by less than 10%, less than 7.5%, less than 5%, less than 2.5%, or less than 1% when shifting between the radially collapsed delivery configuration and the radially expanded deployed configuration, for example. Other configurations are also contemplated.

The stent 10, 110 may be configured to bend into a U-shape and/or an S-shape without kinking. This may provide an advantageous benefit in deployment of the stent 10, 110 as the stent 10, 110 is able to comply with the tortuous anatomy of a patient or subject as the stent 10, 110 is guided toward its terminal destination of deployment within a patient or subject. In other words, when the stent 10, 110 is deployed, for instance through a biliary duct, vessel, or other body lumen, the stent 10, 110 is able to bend along the curvature of the body lumen as the stent 10, 110 is positioned through the body lumen of a patient or subject without causing undue trauma to the tissue of the patient or subject (e.g., ducts, lumens, vessels, organs, organ tissue, lumen walls, vessel walls, etc.). Owing to the flexibility of the wire 20, 120 and flexibility of the covering 40 (e.g., polymeric covering), the stent 10, 110 is also able to revert into its radially expanded shape upon deployment, and maintain patency of a lumen, vessel, duct or the like at its terminal destination of deployment. In other words, the stent 10, 110 may be delivered in a straight configuration for delivery into a patient through a device such as a catheter. As the stent 10, 110 is navigated through the body lumen of a patient or subject, the stent 10 is able to bend with the curves of the body lumen of the patient or subject without causing undue trauma to the body lumen of the patient or subject. Upon reaching the desired area of treatment, the stent 10 may revert to its original radially expanded shape or configuration, or may further conform to the geometry of the desired area of treatment. For instance, and by non-limiting example, the stent 10, 110 may conform to a bend of the bile duct and/or pancreatic duct while maintaining patency in the bile duct or pancreatic duct.

Embodiments herein provide method of forming a stent (e.g., method of forming the stent 10, 110). The method can include receiving a tubular scaffold such as the tubular scaffold 12, 112, described herein. For instance, the tubular scaffold may extend axially from a first end to a second end along a central longitudinal axis; wherein the tubular scaffold is formed from a single wire extending from the first end to the second end; and the single wire forms a plurality of helical windings around the central longitudinal axis, as described herein. In some embodiments, the tubular scaffold can be formed on a mandrel, among other possibilities.

The method can include interweaving a reinforcing filament with the plurality of helical windings of the single wire to form a reinforced framework. For instance, the method can include interweaving the reinforcing filament in an alternating over and under fashion with the plurality of helical windings of the wire, as described herein. In some instances, the method can include interweaving an individual reinforcing filament (e.g., only one reinforcing filament) with the plurality of helical windings of the single wire to form a reinforced framework including an individual reinforcing filament. However, in some instances the method can include interweaving a plurality reinforcing filaments with the helical windings of the single wire to form a reinforced framework including the plurality of reinforcing filaments.

Subsequent to formation of the reinforced framework, the method can include covering the reinforced framework with a polymeric covering to form the stent. In some embodiments, covering the reinforced framework with a polymeric covering may include overlaying the reinforced framework with at least one solid circumferential polymeric sleeve; and heating the solid circumferential polymeric sleeve to cause the solid circumferential polymeric sleeve to reflow and form the polymeric covering. For example, the method can include covering the reinforced framework with a solid polymeric sleeve that overlays all of the reinforcing filament in the reinforced framework. Heating the at least one solid circumferential polymeric covering to a given temperature (e.g., 100 degrees Celsius, etc.) may cause the circumferential sleeve to reflow and thereby form a polymeric covering overlaying at least a portion of the reinforced framework (e.g., such that at least a portion of the reinforced framework is embedded in the polymeric covering). In some embodiments, the polymeric covering (e.g., formed from a circumferential sleeve) can be formed of polyurethane, silicone, or a combination thereof, among other possible materials. For instance, the circumferential polymeric sleeve can be formed of polyurethane. However, in some embodiments the circumferential polymeric sleeve can be formed of silicone.

In some embodiments, the circumferential polymeric sleeve can be manifested as an individual sleeve (e.g., disposed circumferentially about an exterior surface or being disposed about an interior (e.g., intraluminal surface) of the reinforced framework. However, in some embodiments, the circumferential polymeric sleeve can be manifested as two or more sleeves, such as a first sleeve (e.g., a polyurethane sleeve) disposed on an inside surface (e.g., intralumenal surface) of the reinforced framework and a second sleeve (e.g., a silicone sleeve) disposed about an exterior surface of the reinforced framework, among other possibilities.

However, other methods of forming the polymeric covering are possible. For instance, in some embodiments the polymeric covering can for formed by applying a liquid polymeric covering to the reinforced framework. Applying the liquid polymeric covering may take the form of dip coating, spray coating, or otherwise applying a liquid polymeric covering to the reinforced framework such that the liquid polymeric covering subsequently becomes the polymeric covering on the reinforced framework. In any case, at least due to the presence of the reinforced framework (e.g., an interwoven reinforcing filament therein) in conjunction with the other elements of the endoprosthesis, a length of the body portion from the first end to the second end is configured to change by less than 5% when shifting between a collapsed delivery configuration and an expanded deployed configuration, as described herein.

The materials that can be used for the various components of the stent and/or the stent system and the various elements thereof disclosed herein may include those commonly associated with medical devices. For simplicity purposes, the following discussion refers to the stent. However, this is not intended to limit the devices, components, and methods described herein, as the discussion may be applied to other elements, members, components, or devices disclosed herein, such as, but not limited to, the delivery device, the stent, the single wire, the polymeric covering, etc. and/or elements or components thereof.

In some embodiments, the system and/or components thereof may be made from a metal, metal alloy, polymer, a metal-polymer composite, ceramics, combinations thereof, and the like, or other suitable material.

Some examples of suitable polymers may include polytetrafluoroethylene (PTFE), ethylene tetrafluoroethylene (ETFE), fluorinated ethylene propylene (FEP), polyoxymethylene (POM; for example, DELRIN®), polyether block ester, polyurethane, polypropylene (PP), polyvinylchloride (PVC), polyether-ester (for example, ARNITEL®), ether or ester based copolymers (for example, butylene/poly(alkylene ether) phthalate and/or other polyester elastomers such as HYTREL®), polyamide (for example, DURETHAN® or CRISTAMID®), elastomeric polyamides, block polyamide/ethers, polyether block amide (PEBA; for example, PEBAX®), ethylene vinyl acetate copolymers (EVA), silicones, polyethylene (PE), MARLEX® high-density polyethylene, MARLEX® low-density polyethylene, linear low density polyethylene (for example, REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID®), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, polyurethane silicone copolymers (for example, Elast-Eon® or ChronoSil®), biocompatible polymers, other suitable materials, or mixtures, combinations, copolymers thereof, polymer/metal composites, and the like. In some embodiments, the system and/or components thereof can be blended with a liquid crystal polymer (LCP).

Some examples of suitable metals and metal alloys include stainless steel, such as 304 and/or 316 stainless steel and/or variations thereof; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; platinum; palladium; gold; combinations thereof; or any other suitable material.

In at least some embodiments, portions or all of the system and/or components thereof may also be doped with, made of, or otherwise include a radiopaque material. Radiopaque materials are understood to be materials capable of producing a relatively dark image on a fluoroscopy screen or another imaging technique (e.g., ultrasound, etc.) during a medical procedure. This relatively dark image aids the user of the system in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the system to achieve the same result.

In some embodiments, a degree of Magnetic Resonance Imaging (MRI) compatibility is imparted into the system and/or other elements disclosed herein. For example, the system and/or components or portions thereof may be made of a material that does not substantially distort the image and create substantial artifacts (e.g., gaps in the image). Certain ferromagnetic materials, for example, may not be suitable because they may create artifacts in an MRI image. The system or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

In some embodiments, the system and/or other elements disclosed herein may include a fabric material disposed over or within the structure. The fabric material may be composed of a biocompatible material, such a polymeric material or biomaterial, adapted to promote tissue ingrowth. In some embodiments, the fabric material may include a bioabsorbable material. Some examples of suitable fabric materials include, but are not limited to, polyethylene glycol (PEG), nylon, polytetrafluoroethylene (PTFE, ePTFE), a polyolefinic material such as a polyethylene, a polypropylene, polyester, polyurethane, and/or blends or combinations thereof.

In some embodiments, the system and/or other elements disclosed herein may include and/or be formed from a textile material. Some examples of suitable textile materials may include synthetic yarns that may be flat, shaped, twisted, textured, pre-shrunk or un-shrunk. Synthetic biocompatible yarns suitable for use in the present disclosure include, but are not limited to, polyesters, including polyethylene terephthalate (PET) polyesters, polypropylenes, polyethylenes, polyurethanes, polyolefins, polyvinyls, polymethylacetates, polyamides, naphthalene dicarboxylene derivatives, natural silk, and polytetrafluoroethylenes. Moreover, at least one of the synthetic yarns may be a metallic yarn or a glass or ceramic yarn or fiber. Useful metallic yarns include those yarns made from or containing stainless steel, platinum, gold, titanium, tantalum, or a Ni—Co—Cr-based alloy. The yarns may further include carbon, glass, or ceramic fibers. Desirably, the yarns are made from thermoplastic materials including, but not limited to, polyesters, polypropylenes, polyethylenes, polyurethanes, polynaphthalenes, polytetrafluoroethylenes, and the like. The yarns may be of the multifilament, monofilament, or spun types. The type and denier of the yarn chosen may be selected in a manner which forms a biocompatible and implantable prosthesis and, more particularly, a vascular structure having desirable properties.

In some embodiments, the system and/or other elements disclosed herein may include and/or be treated with a suitable therapeutic agent. Some examples of suitable therapeutic agents may include anti-thrombogenic agents (such as heparin, heparin derivatives, urokinase, and PPack (dextrophenylalanine proline arginine chloromethyl ketone)); anti-proliferative agents (such as enoxaparin, angiopeptin, monoclonal antibodies capable of blocking smooth muscle cell proliferation, hirudin, and acetylsalicylic acid); anti-inflammatory agents (such as dexamethasone, prednisolone, corticosterone, budesonide, estrogen, sulfasalazine, and mesalamine); antineoplastic/antiproliferative/anti-mitotic agents (such as paclitaxel, 5-fluorouracil, cisplatin, vinblastine, vincristine, epothilones, endostatin, angiostatin and thymidine kinase inhibitors); anesthetic agents (such as lidocaine, bupivacaine, and ropivacaine); anti-coagulants (such as D-Phe-Pro-Arg chloromethyl ketone, an RGD peptide-containing compound, heparin, anti-thrombin compounds, platelet receptor antagonists, anti-thrombin antibodies, anti-platelet receptor antibodies, aspirin, prostaglandin inhibitors, platelet inhibitors, and tick antiplatelet peptides); vascular cell growth promoters (such as growth factor inhibitors, growth factor receptor antagonists, transcriptional activators, and translational promoters); vascular cell growth inhibitors (such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin); immunosuppressants (such as the “olimus” family of drugs, rapamycin analogues, macrolide antibiotics, biolimus, everolimus, zotarolimus, temsirolimus, picrolimus, novolimus, myolimus, tacrolimus, sirolimus, pimecrolimus, etc.); cholesterol-lowering agents; vasodilating agents; and agents which interfere with endogenous vasoactive mechanisms.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The scope of the disclosure is, of course, defined in the language in which the appended claims are expressed.

What is claimed is:

1. A medical stent, comprising:
a tubular scaffold extending from a first end to a second end along a central longitudinal axis; wherein the tubular scaffold is formed from a single wire extending helically from the first end to the second end; and
wherein the single wire forms a plurality of helical windings around the central longitudinal axis; and
a polymeric covering disposed on the tubular scaffold and spanning gaps between adjacent helical windings of the tubular scaffold.

2. The medical stent of claim 1, further comprising a reinforcing filament extending substantially longitudinally along the tubular scaffold.

3. The medical stent of claim 2, wherein the reinforcing filament has a length that is substantially equal to a length of the tubular scaffold.

4. The medical stent of claim 2, wherein the reinforcing filament is interwoven with the plurality of helical windings of the single wire.

5. The medical stent of claim 4, wherein the reinforcing filament is interwoven in an alternating over and under fashion with the plurality of helical windings of the single wire.

6. The medical stent of claim 2, wherein the reinforcing filament is in direct contact with the plurality of helical windings of the single wire.

7. The medical stent of claim 2, wherein the reinforcing filament is an elongated planar strip.

8. The medical stent of claim 2, wherein the reinforcing filament is formed of polyester, polytetrafluoroethylene, or a combination thereof.

9. The medical stent of claim 2, wherein the reinforcing filament is embedded in the polymeric covering.

10. The medical stent of claim 2, wherein a length of the tubular scaffold from the first end to the second end is configured to change by less than 5% when shifting between a radially collapsed delivery configuration and a radially expanded deployed configuration.

11. The medical stent of claim 1, wherein the single wire forms a double helix having a first helical segment of the single wire extending parallel to a second helical segment of the single wire.

12. A medical stent, comprising:
a tubular scaffold extending from a first end to a second end along a central longitudinal axis; wherein the tubular scaffold is formed from a single wire extending helically from the first end to the second end; and
wherein the single wire forms a plurality of helical windings around the central longitudinal axis;
a polymeric covering disposed on the tubular scaffold and spanning gaps between adjacent helical windings of the tubular scaffold; and
a plurality of longitudinal reinforcing strips extending along the tubular scaffold parallel to the central longitudinal axis.

13. The medical stent of claim 12, wherein the plurality of reinforcing strips is interwoven with the plurality of helical windings of the single wire.

14. The medical stent of claim 12, wherein the plurality of reinforcing strips is embedded in the polymeric covering.

15. The medical stent of claim 12, wherein a length of the tubular scaffold from the first end to the second end is configured to change by less than 5% when shifting between a radially collapsed delivery configuration and a radially expanded deployed configuration.

16. The medical stent of claim 12, wherein the single wire forms a double helix having a first helical segment of the single wire extending parallel to a second helical segment of the single wire.

17. A medical stent, comprising:
a tubular scaffold extending from a first end to a second end along a central longitudinal axis; wherein the tubular scaffold is formed from a single wire extending helically from the first end to the second end; and
wherein the single wire forms a plurality of helical windings around the central longitudinal axis;
a polymeric covering disposed on the tubular scaffold and spanning gaps between adjacent helical windings of the tubular scaffold; and
a plurality of longitudinal reinforcing strips extending along the tubular scaffold;
wherein the plurality of longitudinal reinforcing strips is configured to restrict elongation of the stent by less than 5% when the stent shifts between a radially collapsed delivery configuration and a radially expanded deployed configuration.

18. The medical stent of claim 17, wherein the plurality of reinforcing strips is interwoven with the plurality of helical windings of the single wire.

19. The medical stent of claim 17, wherein the plurality of reinforcing strips is embedded in the polymeric covering.

20. The medical stent of claim 17, wherein the plurality of reinforcing strips is formed of polyester, polytetrafluoroethylene, or a combination thereof.
